(19) United States
(12) Patent Application Publication
(10) Pub. No.: US 2016/0030692 A1
(43) Pub. Date: Feb. 4, 2016

(54) SINGLE-PIECE AEROSOL EXHALATION FILTER AND AN AEROSOL DELIVERY DEVICE INCLUDING THE SAME

(71) Applicant: TELEFLEX MEDICAL INCORPORATED, Morrisville, NC (US)

(72) Inventors: Marc BURK, Menifee, CA (US); Daniel Patrick DWYER, Raleigh, NC (US)

(21) Appl. No.: 14/776,258

(22) PCT Filed: Mar. 12, 2014
(86) PCT No.: PCT/US14/24073

§ 371 Date: Sep. 14, 2015

Related U.S. Application Data
(60) Provisional application No. 61/779,521, filed on Mar. 13, 2013.

(51) Int. Cl. A61M 16/00; A61M 15/00; A61M 16/14; A61M 11/00; A61M 16/10

(52) U.S. Cl. CPC A61M 16/0093; A61M 11/00; A61M 16/1065; A61M 16/14; A61M 15/0021

(57) ABSTRACT

An aerosol delivery device for delivering an aerosol to a patient includes a nebulizer configured to generate an aerosol containing droplets of a liquid medicament, an inhalation conduit defining an inhalation passage, an exhalation conduit defining an exhalation passage, and a single-piece filter connected to the exhalation conduit configured to capture at least some of the droplets of the liquid medicament from a gaseous or fluid medium. The single-piece filter includes a continuous, single-piece filter body having a proximal portion and a distal portion, wherein the proximal portion of the filter body has a proximal end that defines an opening configured to connect to the exhalation conduit and the distal portion of the filter body has a closed distal end.

### CROSS REFERENCE TO RELATED APPLICATION

This application claims the benefit of U.S. Provisional Application No. 61/779,521, filed on Mar. 13, 2013, the disclosure of which is hereby incorporated by reference in its entirety.

### TECHNICAL FIELD

This disclosure generally relates to preventing medicaments produced by an aerosol delivery device from entering the atmosphere. More particularly, this disclosure relates to a single-piece aerosol exhalation filter connected to an exhaust port of an aerosol delivery device for filtering droplets of a liquid medicament from the exhalation of a patient.

### BACKGROUND

Aerosols including medicaments, such as anesthetics, are commonly administered to patients. The aerosols may be administered to the patients using an aerosol delivery device, such as a nebulizer or atomizer The aerosol delivery device can atomize a liquid medicament and the resulting droplets of the liquid medicament will mix with atmospheric air and/or a particular gas from a pressurized gas source for inhalation by the patient. During subsequent exhalation, the patient will breathe out at least some of the droplets of the liquid medicament that were not absorbed by the patient.

Small droplets of medicaments can hang suspended in the atmosphere of, for example, hospital rooms for several hours, putting both medical practitioners and other patients at risk. For example, medical practitioners continuously exposed to even low doses of an anesthetic can lose consciousness or have diminished capacity to provide competent care to the patients under their care. In addition, studies have shown that continuous exposure to bronchodilators has led to occupational asthma in medical practitioners. To prevent medicaments from entering the atmosphere, various exhalation filters have been developed to capture droplets of the medicaments from the exhalation of patients using aerosol delivery devices.

Existing exhalation filters, however, are typically constructed of multiple pieces. For example, many existing exhalation filters include a plastic body that removably receives a porous sheet. Such multi-piece exhalation filters are relatively costly to manufacture, are relatively heavy, require knowledge of assembly of the exhalation filters, and require knowledge of connection of the exhalation filters to their respective aerosol delivery devices. Given the rise in the use of disposable, handheld, small volume aerosol delivery devices, such additional cost, weight, and specialized knowledge of assembly and connection may prevent the use of exhalation filters, resulting in risks associated with the second-hand exposure of the medicaments.

Therefore, a need exists for a single-piece aerosol exhalation filter that is less costly to manufacture, lighter, and easier to use in aerosol delivery devices, such as disposable, handheld, small volume aerosol delivery devices.

### SUMMARY

The foregoing needs are met, to a great extent, by implementations of the single-piece aerosol exhalation filter and the aerosol delivery device including the single-piece aerosol exhalation filter for filtering droplets of a liquid medicament from the exhalation of a patient.

In accordance with one implementation, an aerosol delivery device for delivering an aerosol to a patient includes a nebulizer configured to generate an aerosol containing droplets of a liquid medicament, an inhalation conduit defining an inhalation passage, an exhalation conduit defining an exhalation passage, and a single-piece filter connected to the exhalation conduit configured to capture at least some of the droplets of the liquid medicament from a gaseous or fluid medium. The inhalation passage is configured to allow passage of the aerosol generated by the nebulizer to the patient. The exhalation passage is configured to allow passage of a gaseous or fluid medium exhaled from the patient where the gaseous or fluid medium includes droplets of the liquid medicament. The single-piece filter includes a continuous, single-piece filter body having a proximal portion and a distal portion, wherein the proximal portion of the filter body has a proximal end that defines an opening configured to connect to the exhalation conduit and the distal portion of the filter body has a closed distal end.

In some implementations, the single-piece filter can have a generally tubular shape and the opening defined by the proximal end of the filter body can have a circular cross section. The shape of the cross section of the exhalation conduit can be the same as the shape of the cross-section of the opening defined by the proximal end of the filter body. The opening of the proximal portion of the filter body can have a diameter between 15 mm and 30 mm. The single-piece filter can have an internal volume between five mL and 200 mL, and an axial length between 7.5 cm and 30 cm.

In some implementations, the single-piece filter can be connected to the exhalation conduit through a friction fit. The inhalation passage can be fluidly coupled to the nebulizer and the exhalation passage can be fluidly coupled to the inhalation passage. An exhaust port can be located at one end of the exhalation conduit. The single-piece filter can be made of a microporous material. The microporous material can be capable of filtering at least 95% of airborne particles with aerodynamic diameters of 0.3 micrometres or greater. The gaseous or fluid medium can be a patient's breath.

In accordance with another implementation, an aerosol delivery device for delivering an aerosol to a patient includes an exhalation conduit defining an exhalation passage and a single-piece filter connected to the exhalation conduit. The exhalation passage is configured to allow passage of a gaseous or fluid medium exhaled from the patient, where the gaseous or fluid medium includes droplets of a liquid medicament. The single-piece filter is configured to capture at least some of the droplets of the liquid medicament from the gaseous or fluid medium. The single-piece filter includes a continuous, single-piece filter body having a proximal portion and a distal portion. The proximal portion of the filter body has a proximal end that defines an opening configured to connect to the exhalation conduit and the distal portion of the filter body has a closed distal end.

In accordance with yet another implementation, a single-piece exhalation filter includes a continuous, single-piece filter body having a proximal portion and a distal portion. The proximal portion of the filter body has a proximal end that defines an opening configured to connect to an exhalation conduit of an aerosol delivery device, and the distal portion of the filter body has a closed distal end. The single-piece exhalation filter is configured to capture a least some droplets of a liquid medicament from a gaseous or fluid medium.

Certain implementations of the single-piece aerosol exhalation filter and the aerosol delivery device including the single-piece aerosol exhalation filter have been outlined so that the detailed description below may be better understood. There are, of course, additional implementations that will be described below and which will form the subject matter of the claims.

In this respect, before explaining at least one implementation in detail, it is to be understood that the single-piece aerosol exhalation filter and the aerosol delivery device including the single-piece aerosol exhalation filter are not limited in their application to the details of construction and to the arrangements of the components set forth in the following description or illustrated in the drawings. Also, it is to be understood that the phraseology and terminology employed herein, as well as the Abstract, are for the purpose of description and should not be regarded as limiting.

As such, those skilled in the art will appreciate that the conception upon which this disclosure is based may readily be utilized as a basis for the designing of other structures, methods, and systems for carrying out the several purposes of the single-piece aerosol exhalation filter and the aerosol delivery device including the single-piece aerosol exhalation filter. It is understood, therefore, that the claims include such equivalent constructions insofar as they do not depart from the spirit and scope of the present application.

### BRIEF DESCRIPTION OF THE DRAWINGS

FIG. 1 is a perspective view illustrating an exemplary aerosol delivery device including a single-piece aerosol exhalation filter.

### DETAILED DESCRIPTION

Implementations of the single-piece aerosol exhalation filter and the aerosol delivery device including the single-piece aerosol exhalation filter are described with reference to the drawings, in which like reference numerals refer to like parts throughout.

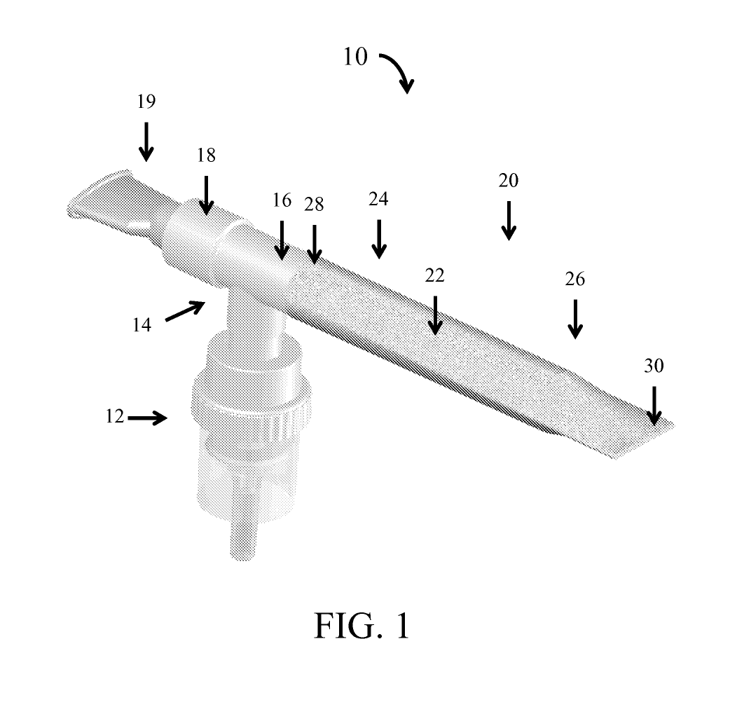

Referring to FIG. 1, an aerosol delivery device 10 including a single-piece aerosol exhalation filter 20 is illustrated. The aerosol delivery device 10 includes a nebulizer 12, a connecter 14 having a tee-shape defining an exhalation conduit 16 and an inhalation conduit 18 at opposite ends of the connecter 14, a mouthpiece 19 connected to the inhalation conduit 18, and the single-piece aerosol exhalation filter 20 connected to the exhalation conduit 16. An exhaust port can be located at the distal end of the exhalation conduit 16 and is covered by the single-piece aerosol exhalation filter 20. The exhalation conduit 16 defines an exhalation passage or lumen while the inhalation conduit 18 defines an inhalation passage or lumen. The inhalation passage can be in fluid communication with the nebulizer 12 and the exhalation passage can be in fluid communication with the inhalation passage.

The single-piece aerosol exhalation filter 20 includes a continuous, single-piece body 22 having a proximal portion 24 and a distal portion 26. The single-piece aerosol exhalation filter 20 is made completely of a macroporous or microporous material that captures the droplets of the liquid medicament from the patient's breath, thereby filtering the patient's breath to prevent the droplets from entering the atmosphere. The proximal portion 24 has a proximal end 28 that defines an opening configured to connect to the exhalation conduit 16. The distal portion 26 has a closed distal end 30 that does not allow any gaseous or fluid medium to escape without passing through the single-piece aerosol exhalation filter 20.

The nebulizer 12 produces an aerosol by generating droplets of a liquid medicament that are mixed with pressurized air and/or atmospheric air. The pressurized air can be, for example, oxygen or the like used for atomization of the liquid medicament. The aerosol travels through the connecter 14 defining the inhalation conduit 18 and the mouthpiece 19 to deliver the medicament to the patient during inhalation. As the patient exhales, the patient's breath, including droplets of the liquid medicament that were not absorbed by the patient, travels through the mouthpiece 19, the connecter 14 defining the inhalation conduit 18 and the exhalation conduit 16, and to the single-piece aerosol exhalation filter 20. The droplets of the liquid medicament will then be captured by the single-piece aerosol exhalation filter 20 while the gases of the patient's breath will pass through the single-piece aerosol exhalation filter 20 to the atmosphere.

To allow the patient to breathe without restriction, the material used to form the single-piece body 22 may allow low resistance gas flow in both directions. In particular, the single-piece aerosol exhalation filter 20 may have sufficient dimensions and resistance to enable a patient with weak or compromised breathing to breathe through the aerosol delivery device 10 for up to 60 minutes without fatigue. In some implementations, the flow resistance through the single-piece aerosol exhalation filter 20 may be less than, for example, five cm.H₂O when 60 LPM is directed through the single-piece aerosol exhalation filter 20.

In addition to allowing low resistance gas flow, however, the single-piece aerosol exhalation filter 20 can be made of a material with sufficient density to trap a majority of the droplets of the liquid medicament in the patient's breath. In some implementations, the material used to form the single-piece body 22 may trap over 90% and, preferably, over 99% of the droplets of the liquid medicament.

If the material is used to form the single-piece body 22 is microporous, the material can have pores with diameters between two nanometers and 50 nanometers. The pore size of the material used to form the single-piece body 22 should be small enough to capture the droplets of the liquid medicament within the material or to prevent the droplets from passing through the material.

In some implementations, the material used to form the single-piece body 22 can have, for example, an N95 rating capable of filtering at least 95% of airborne particles with aerodynamic diameters of 0.3 micrometres or greater, an N99 rating capable of filtering at least 99% of airborne particles, or an N100 rating capable of filtering at least 99.7% of airborne particles, i.e., High-Efficiency Particulate Air (“HEPA”) rating. Preferably, the material used to form the single-piece body 22 can be capable of filtering at least 95% of the airborne particles. The material may or may not be oil resistant.

The material may include strands of woven or non-woven material which can be layered to acquire a desired filtration density of, for example, between 30 and 300 g/cm³. The strands can be thermoplastic strands made of polypropylene, polyethylene, or the like. The material can have sufficient mechanical strength to support the weight of the single-piece aerosol exhalation filter 20 without additional support. In some implementations, one layer of the material can be used to form the single-piece body 22, whereas in other implementations, multiple layers of the material can be wrapped to form the single-piece body 22.

As illustrated in FIG. 1, the proximal portion 24 of the single-piece body 22 can have a generally tubular, i.e., cylindrical, shape with a circular cross section. In some implementations, however, the proximal portion 24 of the single-piece body 22 can have an elliptical cross section, a square cross section, a rectangular cross section, a pentagon cross section, or the like. The cross section of the proximal portion 24 of the single-piece body 22 will depend on the cross section of the exhalation conduit 16, such that the cross section of the proximal portion 24 of the single-piece body 22 and the cross section of the exhalation conduit 16 can preferably be the same. By having the same cross section shape, the patient's breath will pass from the connecter 14 to the single-piece aerosol exhalation filter 20 without escaping and a friction fit of sufficient strength is assured.

The single-piece body 22 may have a shape that can support itself, such that the single-piece aerosol exhalation filter 20 does not require additional support, such as a frame. By not requiring an additional support, the single-piece aerosol exhalation filter can be manufactured more economically as a single piece. In addition to being self-supporting, a tubular shape can also be manufactured more economically relative to other shapes. Moreover, because many aerosol delivery devices have an exhalation conduit with a circular cross section, the tubular shape of the single-piece aerosol exhalation filter 20 enables it to be used in combination with a variety of aerosol delivery devices.

The nebulizer 12, connecter 14, and mouthpiece 19 can be made of one or more substantially rigid materials such as, plastic including, for example, acrylic, polyethylene, polyamide, or polyvinyl chloride; metals including, for example, steel or aluminum; and/or combinations thereof.

The single-piece aerosol exhalation filter 20 can be connected to the exhaust port of the exhalation conduit 16 using one or more of a variety of different connections means, such as, for example, friction fit, adhesives, tongue and grooves, rubber bands, zip ties, nails, screws, clips, or the like. In preferred implementations, the single-piece aerosol exhalation filter 20 is connected to the exhaust port by friction fit. For example, where the outer diameter of the single-piece aerosol exhalation filter 20 is equal to the outer diameter of the exhalation conduit 16, the outer diameter of the exhaust port can be smaller than the outer diameter of the exhalation conduit 16. In particular, the outer diameter of the exhaust port can be equal to the outer diameter of the exhalation conduit 16 minus twice the thickness of the body of the single-piece aerosol exhalation filter 20. In another example, the inner diameter of the single-piece aerosol exhalation filter 20 can be equal to the outer diameter of the exhaust port to enable a friction fit between the two.

In some implementations, the exhaust port at the distal end of the exhalation conduit 16 can have a circular cross section with an outer diameter between 15 and 30 mm and, preferably, about 22 mm. As such, the opening at the proximal end 28 of the single-piece aerosol exhalation filter 20 can also have a circular cross section with an inner diameter of about 22 mm to enable a friction fit connection of the single-piece aerosol exhalation filter 20 to the exhalation conduit 16.

The internal volume of the single-piece aerosol exhalation filter 20 should be sufficient to collect the droplets of the liquid medicament that would otherwise be wasted and harmful to others. In some implementations, the internal volume of the single-piece aerosol exhalation filter 20 can be between five mL and 200 mL. Testing has shown that an internal volume of about 50 mL is sufficient to collect the droplets of the liquid medicament from a patient's breath.

The axial length of the single-piece aerosol exhalation filter 20 can depend on the desired internal volume of the single-piece aerosol exhalation filter 20 and the diameter of the exhaust port of the aerosol delivery device 10. In some implementations, the axial length of the single-piece aerosol exhalation filter 20 can be between 7.5 cm and 30 cm. Preferably, however, to manufacture a single-piece aerosol exhalation filter 20 with an internal volume of about 50 mL having an opening defined by the proximal end 28 with a diameter of 22 mm, the axial length may be about 15 cm.

In some implementations, the closed distal end 30 of the single-piece aerosol exhalation filter 20 can be formed by bonding the opposite sides of the distal portion 26 together after the tubular single-piece body 22 is formed. The opposite sides of the distal portion 26 can be bonded together using an adhesive, heat fusion, chemical fusion, or the like. In other implementations, the closed distal end 30 can be originally formed as a closed end during manufacturing. For example, the single-piece aerosol exhalation filter 20 can be extruded with a closed distal end 30.

The many features and advantages of the single-piece aerosol exhalation filter 20 are apparent from the detailed specification, and thus, the claims cover all such features and advantages within the scope of this application. Further, numerous modifications and variations are possible.

For example, in addition to filtering the droplets of the liquid medicament, the single-piece aerosol exhalation filter 20 can also be configured to filter bacteria molecules and viruses present in the patient's breath. In particular, the material forming the single-piece body 22 can be selected to have suitable pore sizes effective for filtering the droplets of the liquid medicament, as well as bacteria and viruses.

In another example, the aerosol delivery device 10 can include one or more one-way valves in the exhalation conduit 16 and/or the inhalation conduit 18 to block the fluid communication of one or more passages. For example, the fluid communication between the nebulizer 12 and the inhalation passage can be blocked during exhalation using a one-way valve in the connecter 14.

As such, it is not desired to limit the single-piece aerosol exhalation filter 20 to the exact construction and operation described and illustrated, and accordingly, all suitable modifications and equivalents may fall within the scope of the claims.

What is claimed is:

1. An aerosol delivery device for delivering an aerosol to a patient, the aerosol delivery device comprising:
a nebulizer configured to generate an aerosol containing droplets of a liquid medicament;
an inhalation conduit defining an inhalation passage, the inhalation passage configured to allow passage of the aerosol generated by the nebulizer to the patient;
an exhalation conduit defining an exhalation passage, the exhalation passage configured to allow passage of a gaseous or fluid medium exhaled from the patient, the gaseous or fluid medium comprising droplets of the liquid medicament; and
a single-piece filter connected to the exhalation conduit, the single-piece filter configured to capture at least some of the droplets of the liquid medicament from the gaseous or fluid medium, wherein:
the single-piece filter comprises a continuous, single-piece filter body having a proximal portion and a distal portion,
the proximal portion of the filter body has a proximal end that defines an opening configured to connect to the exhalation conduit, and
the distal portion of the filter body has a closed distal end.

2. The aerosol delivery device of claim 1, wherein the single-piece filter has a generally tubular shape.

3. The aerosol delivery device of claim 1, wherein the opening defined by the proximal end of the filter body has a circular cross section.

4. The aerosol delivery device of claim 1, wherein the opening defined by the proximal end of the filter body has a diameter between 15 mm and 30 mm.

5. The aerosol delivery device of claim 1, wherein the single-piece filter is connected to the exhalation conduit through a friction fit.

6. The aerosol delivery device of claim 1, wherein:
the inhalation passage is fluidly coupled to the nebulizer, and
the exhalation passage is fluidly coupled to the inhalation passage.

7. The aerosol delivery device of claim 1, wherein the single-piece filter is made of a microporous material.

8. The aerosol delivery device of claim 7, wherein the microporous material is capable of filtering at least 95% of airborne particles with aerodynamic diameters of 0.3 micrometres or greater.

9. The aerosol delivery device of claim 1, wherein the single-piece filter has an internal volume between five mL and 200 mL.

10. The aerosol delivery device of claim 1, wherein the single-piece filter has an axial length between 7.5 cm and 30 cm.

11. The aerosol delivery device of claim 1, further comprising an exhaust port located at one end of the exhalation conduit, wherein the opening of the proximal portion of the filter body is configured to connect to the exhaust port.

12. The aerosol delivery device of claim 1, wherein a shape of a cross section of the exhalation conduit is the same as a shape of a cross-section of the opening of the proximal portion of the filter body.

13. The aerosol delivery device of claim 1, wherein the gaseous or fluid medium is a patient's breath.

14. An aerosol delivery device for delivering an aerosol to a patient, the aerosol delivery device comprising:
an exhalation conduit defining an exhalation passage, the exhalation passage configured to allow passage of a gaseous or fluid medium exhaled from the patient, the gaseous or fluid medium comprising droplets of a liquid medicament; and
a single-piece filter connected to the exhalation conduit, the single-piece filter configured to capture at least some of the droplets of the liquid medicament from the gaseous or fluid medium, wherein:
the single-piece filter comprises a continuous, single-piece filter body having a proximal portion and a distal portion,
the proximal portion of the filter body has a proximal end that defines an opening configured to connect to the exhalation conduit, and the distal portion of the filter body has a closed distal end.

15. An single-piece exhalation filter, comprising a continuous, single-piece filter body having a proximal portion and a distal portion, the proximal portion of the filter body having a proximal end that defines an opening configured to connect to an exhalation conduit of an aerosol delivery device, and the distal portion of the filter body having a closed distal end, wherein the single-piece exhalation filter is configured to capture at least some droplets of a liquid medicament from a gaseous or fluid medium.
